(19) United States
(12) Patent Application Publication
(10) Pub. No.: US 2024/0271196 A1
(43) Pub. Date: Aug. 15, 2024

(54) NUCLEIC ACID SEQUENCE ANALYSIS

(71) Applicant: Arizona Board of Regents on behalf of Arizona State University, Scottsdale, AZ (US)

(72) Inventors: Stephan Baehr, Scottsdale, AZ (US); Jean-Francois Gout, Starkville, MS (US); Michael Lynch, Mesa, AZ (US)

(21) Appl. No.: 18/435,731

(22) Filed: Feb. 7, 2024

Related U.S. Application Data
(60) Provisional application No. 63/483,650, filed on Feb. 7, 2023.

(51) Int. Cl. C12Q 1/6844; C12Q 1/44; C12Q 1/6869

(52) U.S. Cl. CPC C12Q 1/6844; C12Q 1/44; C12Q 1/6869

(57) ABSTRACT

The present disclosure provides methods related to nucleic acid amplification and sequencing. In particular, the present disclosure provides methods for amplifying and sequencing double stranded nucleic acids using a high yield and resolution circularizing-sequencing method.

### CROSS-REFERENCE TO RELATED APPLICATIONS

This application claims the benefit of U.S. Provisional Application No. 63/483,650, filed Feb. 7, 2023, the content of which is herein incorporated by reference in its entirety.

### FIELD

The present disclosure provides methods related to nucleic acid amplification and sequencing. In particular, the present disclosure provides methods for amplifying and sequencing nucleic acids using a high yield and high resolution circularizing-sequencing method.

### BACKGROUND

Nucleic acid mutation is the basis of all heritable biological variation. Some genetic variation gives rise to beneficial traits, most mutations are neutral or nearly neutral, and some mutations result in severely deleterious phenotypes. Nucleic acid mutations are commonly thought to affect evolutionary trajectories of phenotypes over millions of years, but advances in the last century have shown that evolution can occur over hours, days, or years. For example, the emergence of organisms resistant to herbicides, pesticides, and antibiotics threaten great triumphs of biotechnology and medicine. Rapid evolution of the common cold and novel viruses such as COVID-19 decrease human productivity and have proven their ability to constrict the global economy. Accumulated mutations in somatic cells cause cancer, help cancer to evade anticancer treatments, and broadly degrade the efficiency of bodily functions in aging. Pinpointing novel mutations in populations of cells is therefore of particular interest to the study of diverse human-related subjects, from viral evolution to personalized medicine and predictive medicine. Precise measurement of nucleic acid mutations is therefore desirable to rapidly detect and analyze low frequency mutations in a population of cells. Precise measurement of mutations by high throughput sequencing has been hindered by sequencing error rates, which occur at rates approximately 1×10⁻² to 1×10⁻³ per base sequenced.

### SUMMARY

The present disclosure provides methods for amplifying and/or sequencing nucleic acids.

In some embodiments, the methods comprise amplifying double or single stranded nucleic acids. In some embodiments, the methods comprise fragmenting isolated nucleic acids to generate nucleic acid fragments; when the nucleic acid is double stranded, denaturing the double stranded nucleic acid fragments to form single strand fragments; circularizing the fragments; and amplifying circularized fragments with rolling circle amplification. In some embodiments, the methods produce at least about 1 microgram of amplification products. In some embodiments, the methods produce amplification products at a mutation rate with a sequencing resolution floor of at least about 1×10⁻⁸ per base. In some embodiments, the methods produce amplification products at a mutation rate with a sequencing resolution floor of at least about 1×10⁻⁷ per base.

In some embodiments, the fragmenting comprises contacting the isolated nucleic acids with a fragmentation enzyme (e.g., nucleases). In some embodiments, the fragmentation enzyme is fragmentase, which is a nickase paired to an endonuclease. In some embodiments, fragmentation enzyme is micrococcal nuclease (MNase). In some embodiments, the reaction temperature of the fragmentation enzyme is less than 50 degrees Celsius. In some embodiments, the reaction temperature of the fragmentation enzyme is from 4 to 42 degrees Celsius. In some embodiments, the reaction temperature of the fragmentation enzyme is from 25 to 37 degrees Celsius.

In some embodiments, the denaturing comprises treating the nucleic acid fragments under conditions of alkaline pH. In some embodiments, the alkaline pH is greater than 11. In some embodiments, the alkaline pH is about 12.5.

In some embodiments, the circularizing comprises contacting the fragments with a ligase. In some embodiments, the ligase is an RNA ligase. In some embodiments, the circularizing is completed at 25 degrees Celsius. In some embodiments, the circularizing does not comprise contacting the fragments with DNA repair enzymes. In some embodiments, the ligation time is 30 minutes at 25 degrees Celsius. In some embodiments, the ligation time is 2 hours at 25 degrees Celsius.

In some embodiments, the methods do not comprise size selection of the fragments. In some embodiments, the methods comprise size selection of the fragments.

In some embodiments, the methods further comprise treating nucleic acid fragments with a nucleotide kinase. In some embodiments, the methods further comprise removing non-circularized fragments prior to amplifying. In some embodiments, the methods further comprise extracting the nucleic acid without high temperature or phenol-chloroform extraction.

In some embodiments, the nucleic acid fragments are less than 150 basepairs (bp) or nucleotides (nt). In some embodiments, the nucleic acid fragments are 40-80 bp or nt.

In some embodiments, the rolling circle amplification is primed with random primers. In some embodiments, the rolling circle amplification comprises incubating the circularized single strand fragments with a buffer having an EDTA concentration of 1 uM. In some embodiments, the rolling circle amplification lacks a primer annealing step. In some embodiments, the rolling circle amplification lacks DNA repair enzymes.

In some embodiments, the isolated nucleic acid is present at an amount of less than 250 ng. In some embodiments, the isolated nucleic acid is present at an amount of less than 125 ng.

In some embodiments, the isolated nucleic acid is from a mammal, a plant, or a microorganism. In some embodiments, the isolated nucleic acid is DNA. In some embodiments, the isolated nucleic acid is genomic DNA.

In some embodiments, the genomic DNA is obtained from a biological sample from a subject or microorganism. In some embodiments, the genomic DNA is obtained from a diseased or suspected of being diseased tissue or cell population. In some embodiments, the tissue or cell population is cancerous or suspected of being cancerous.

In some embodiments, the methods further comprise sequencing the amplification products. In some embodiments, the sequencing comprises preparing a library of amplification products, wherein each amplification product has at least one adaptor. In some embodiments, the sequencing further comprises amplifying and sequencing the library of amplification products.

In some embodiments, the methods further comprise detecting nucleotide variations or mutations (e.g., nucleotide substitutions, insertions, and/or deletions) in the amplification products or the library of amplification products. In some embodiments, the methods further comprise identifying disease- or phenotype-associated sequence variations. In some embodiments, the methods further comprise determining mutation rates (e.g., rates of nucleotide substitutions, insertions, and/or deletions).

In some embodiments, the methods comprise amplifying single stranded nucleic acids. In some embodiments, the methods comprise fragmenting isolated single stranded nucleic acids to generate a plurality of single stranded nucleic acid fragments; circularizing the plurality of single strand fragments; and amplifying circularized single strand fragments with rolling circle amplification. In some embodiments, the rolling circle amplification comprises rolling circle reverse transcription.

Other aspects and embodiments of the disclosure will be apparent in light of the following detailed description.

### BRIEF DESCRIPTION OF THE DRAWINGS

FIG. 1 is a schematic of the workflow of circle-sequencing and mu-seq. Genomic DNA is taken and modified into a sequencing library with built-in redundancy for every base. In-vitro damage of DNA can ostensibly occur at every step of the protocol. True mutations are distinct from polymerase and sequencing error. The DNA rolling circle protocol begins with genomic DNA, which is fragmented. After fragmentation, the DNA is end-repaired, melted, circularized to itself, and then undergoes rolling circle amplification. True mutations (yellow circles) are present at the beginning and throughout. These mutations end up on some circles, which when amplified, produce a mutation in the exact same location within tandem repeats. Polymerase errors (purple circles) emerge, as do sequencing errors (blue circle). However, in contrast to true mutations, these errors occur randomly, and can be bioinformatically discerned from one another. The only risk to the protocol are in vitro DNA damage events that occur prior to rolling circle amplification, as they will have the same repeat pattern.

FIG. 2 is mu-seq mutation rate spectra of MutL MMR-E coli PFM5 (top), and from MA/WGS (Lee et al., 2012, Proc. Natl. Acad. Sci. 109, E2774-E2783, bottom). The mutation spectrum of DNA mutation is the relative abundancies of different kinds of mutations, as annotated on the X axis.

FIG. 3 is a list of mutation rate estimates. Mutation estimates among MA studies are similar. Base pair substitutions (BPS) counted are divided by the number of sites in a genome, ˜4.6 m in E coli, and the number of generations of growth.

FIG. 4 is a summary of circle-sequencing mutation rate. A comparison of circle-seq mutation rate estimates of MMR-E. coli, in comparison to published estimates of MA experiments (left) and the original 2013 circle-sequencing paper (Lou et al., 2013, Proc. Natl. Acad. Sci. 110(49):19872-19877), which are based upon yeast genomic DNA. E.c.; E. coli. MMR-: Mismatch-Repair Deficient. Error bars: Standard Error of the Mean.

FIG. 5 is mutation spectra ratios of MMR-E. coli, comparison of Liquid MA vs. Circle-sequencing (RCA). A ratio comparison shows that although there is some signal of the Liquid MA detected, there is also a significant contribution of mutations that are not expected. Approximately two-thirds of the mutations detected by Circle-sequencing are not present in the Liquid MA.

FIGS. 6A and 6B show the impact of ultrasonication upon circle-sequencing. FIG. 6A is an exemplary histogram of repeat lengths generated from circle-sequencing by sonication. FIG. 6B is the mutation spectrum of sonicated samples vs. enzymatic fragmentation. The DNB-seq MMR-spectra here are the same as the 2023 DNA RCA spectra of FIG. 5, for comparison. Error bars in FIG. 6B: standard error of the mean.

### DETAILED DESCRIPTION

Embodiments of the present disclosure include systems and methods related to nucleic acid amplification and sequencing. In particular, the present disclosure provides methods which result in high yield (e.g., greater than 1 microgram) of amplification products and high resolution (e.g., a mutation rate with a sequencing resolution floor of at least about 1×10⁻⁸ per base).

Precise measurement of nucleic acid mutations using high-throughput sequencing is hindered by the error-prone nature of sequencing machines, which generally have error rates between 1 in 100 bases or 1 in 1000 bases. It is difficult to measure mutations which occur once in a million bases, when your background noise signal occurs every thousand bases or less. To get around this limitation, researchers have employed several techniques. In the evolutionary field, mutation rates were first assessed in the Luria-Delbruck fluctuation test, which uses a phenotypic reporter of viral or antibiotic resistance to identify instances of mutation, or some other reporter assay such as loss rate of GFP signal. Presently, any mutation rate can be empirically measured by a mutation accumulation experiment employing recurrent single-cell bottlenecks, followed by high throughput sequencing, at the significant cost of time and labor. In the perspective of tumor evolution, recent advances have grown stem cells in plates, and have used laser-capture microdissection to create duplicate or triplicate measurements of the same sample. DNA barcoding paired with ultra-deep sequencing is employed to detect rare mutations, and Duplex-seq is yet another method developed to get around high sequencing error rates. Though all of these methods appear to work at some level, each has their drawbacks, including high labor costs or low throughput.

When introduced circle-sequencing appeared to have exemplary attributes with regard to labor cost and throughput. When it was published it claimed the lowest potential resolution floor to sequencing, by employing tandem repeats and DNA repair enzymes. These innovations sought to ameliorate DNA damage induced during the DNA extraction and library preparation steps prior to sequencing. However, the initially observed error rate using the method, 7.6×10⁻⁶, was replicated for organisms known to have mutation rates several orders of magnitude lower, for example MMR- or Wild-Type E coli strains.

Described herein is a technique of analyzing nucleic acid sequences and measuring mutations and mutation rates, “μ-seq,” or “mu-seq”, which refines and revamps circle-sequencing methods. Mu-seq can use as little as 125 ng of genomic DNA and obtains mutation rate estimates with a sequencing resolution floor of at least ˜1×10⁻⁸ per base. Mu-seq accurately identified the mutation spectrum of MMR-E coli, although the mutation rate was determined as ˜3.85×10⁻⁹ per base per generation, almost an order of magnitude below the estimated mutation rate from mutation accumulation experiments. However, other experimental results support this range of mutation rate in liquid growth cultures, indicative of a potential oversight in the importance of environment in the measurement of bacterial mutation rates. Mu-seq is thus capable of detecting a mutation rate of 4 mutations in 1 billion bases per site per generation, distinct from experimental noise, and as such, demonstrates the ability to calculate DNA mutation rates significantly lower than those expected in viruses, somatic cells, or cancer cells over time.

In comparison to other methods, mu-seq is streamlined, capable of being entirely automated, uses less expensive reagents, and has an acceptable resolution level for measuring many biological mutation rates. Duplex-seq appears to have roughly the same resolution floor of 1×10⁻⁸, but does not have high yield. Furthermore, the bioinformatics of discarding mutations is somewhat arbitrary. Laser-capture microdissection triplets have the same sequencing cost and the same resolution, perhaps even lower, but the method is very laborious. Mu-seq requires a single biopsy of tissue, while laser-capture microdissection requires 3 exquisitely tiny samples. DNA barcoding+ultradeep sequencing faces a similar problem as duplex-seq; high cost, low yield, and arbitrary bioinformatic sifting rules.

Methods for high-resolution sequencing and detection of mutations in a nucleic acids find use in many areas. Mu-seq is suitable for sequencing tissue biopsies, be they cancerous or not. Sequencing these tissues facilitates understanding of the genomic variation present in a tissue. Mu-seq may also be used to predict what mutations may arise and fall in microbial (e.g., viruses, bacteria, etc.) populations, in normal growth and in response to antimicrobial treatments (e.g., therapeutics, cleansers, etc.). DNA mutation accumulation may be used as a proxy or biomarker for cell age or tissue age, useful for stem cell technology, and a high yield and high resolution for detecting mutations, like mu-seq, is necessary for accurate determination.

Section headings as used in this section and the entire disclosure herein are merely for organizational purposes and are not intended to be limiting.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

As noted herein, the disclosed embodiments have been presented for illustrative purposes only and are not limiting. Other embodiments are possible and are covered by the disclosure, which will be apparent from the teachings contained herein. Thus, the breadth and scope of the disclosure should not be limited by any of the above-described embodiments but should be defined only in accordance with claims supported by the present disclosure and their equivalents. Embodiments of the subject disclosure may also include methods that may further include any and all elements from any other disclosed methods. In other words, elements from one or another disclosed embodiments may be interchangeable with elements from other disclosed embodiments. Moreover, some further embodiments may be realized by combining one and/or another feature disclosed herein with methods and one or more features thereof, disclosed in materials incorporated by reference. In addition, one or more features/elements of disclosed embodiments may be removed and still result in patentable subject matter (and thus, resulting in yet more embodiments of the subject disclosure). Furthermore, some embodiments correspond to methods which specifically lack one and/or another element, structure, and/or steps (as applicable), as compared to teachings of the prior art, and therefore represent patentable subject matter and are distinguishable therefrom (e.g., claims directed to such embodiments may contain negative limitations to note the lack of one or more features prior art teachings).

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, e.g., elements that are conjunctively present in some cases and disjunctively present in other cases. Multiple elements listed with “and/or” should be construed in the same fashion, e.g., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, e.g., the inclusion of at least one, but also including more than one, of a number or list of elements, and, optionally, additional unlisted items. Only terms clearly indicated to the contrary, such as “only one of” or “exactly one of” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of” “only one of” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used herein, a “nucleic acid” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide or ribonucleotide, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single stranded or double stranded form, including homoduplex, heteroduplex, and hybrid states. Hence, the term “nucleic acid” may also encompass a chain comprising non-natural nucleotides, modified nucleotides, and/or non-nucleotide building blocks that can exhibit the same function as natural nucleotides (e.g., “nucleotide analogs”); further, the term “nucleic acid” as used herein refers to an oligonucleotide, nucleotide or polynucleotide, and fragments or portions thereof, and to DNA or RNA of genomic or synthetic origin, which may be single or double stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of proteins, nucleic acids, or compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods provided herein, the mammal is a human.

The term “contacting” as used herein refers to bring or put in contact, to be in or come into contact. The term “contact” as used herein refers to a state or condition of touching or of immediate or local proximity.

As used herein, the term “sample” is used in its broadest sense. In one sense, it is meant to include a specimen obtained from any source, including biological samples. Biological samples may be obtained from a subject (e.g., animals (including humans)) and encompass fluids, solids, tissues, and gases. Such examples are not however to be construed as limiting the sample types. Preferably, a sample is a fluid sample such as a liquid sample. Examples of liquid samples that may be assayed include bodily fluids (e.g., blood, serum, plasma, saliva, urine, ocular fluid, semen, sputum, sweat, tears, and spinal fluid), samples from home, municipal, or industrial water sources, runoff water, or sewage samples; and food samples (e.g., milk, beer, juice, or wine). Viscous liquid, semisolid, or solid specimens may be used to create liquid solutions, eluates, suspensions, or extracts that can be samples. For example, throat or genital swabs may be suspended in a liquid solution to make a sample. Samples can include a combination of liquids, solids, gasses, or any combination thereof (e.g., a suspension of lysed or unlysed cells in a buffer or solution). Samples can comprise biological materials, such as cells, microbes, organelles, and biochemical complexes. Liquid samples can be made from solid, semisolid, or highly viscous materials, such as soils, fecal matter, tissues, organs, biological fluids, or other samples that are not fluid in nature. For example, solid or semisolid samples can be mixed with an appropriate solution, such as a buffer, a diluent, and/or extraction buffer. The sample can be macerated, frozen and thawed, or otherwise extracted to form a fluid sample. Residual particulates may be removed or reduced using conventional methods, such as filtration or centrifugation.

“Test sample,” “sample from a subject,” “biological sample,” and “patient sample” as used interchangeably herein may be a sample of blood, such as whole blood (including for example, capillary blood, venous blood, dried blood spot, etc.), tissue, urine, serum, plasma, amniotic fluid, an anal sample (such as an anal swab specimen), lower respiratory specimens such as, but not limited to, sputum, endotracheal aspirate or bronchoalveolar lavage, nasal mucus, cerebrospinal fluid, placental cells or tissue, endothelial cells, leukocytes, or monocytes. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

A variety of cell types, tissue, or bodily fluid may be utilized to obtain a sample. Such cell types, tissues, and fluid may include sections of tissues such as biopsy and autopsy samples, oropharyngeal specimens, nasopharyngeal specimens, nasal mucus specimens, frozen sections taken for histologic purposes, blood (such as whole blood, dried blood spots, etc.), plasma, serum, red blood cells, platelets, an anal sample (such as an anal swab specimen), interstitial fluid, cerebrospinal fluid, etc. Cell types and tissues may also include lymph fluid, cerebrospinal fluid, or any fluid collected by aspiration. A tissue or cell type may be provided by removing a sample of cells from a human and a non-human animal, but can also be accomplished by using previously isolated cells (e.g., isolated by another person, at another time, and/or for another purpose). Archival tissues, such as those having treatment or outcome history, may also be used.

### 2. METHODS

Embodiments of the present disclosure include methods for amplifying a nucleic acid. The methods may comprise one or more or all of: fragmenting isolated nucleic acids to generate a plurality of nucleic acid fragments, when the nucleic acid is double stranded, denaturing the double stranded nucleic acid to generate a plurality of single strand fragments, circularizing the plurality of fragments (e.g., by contacting the single strand fragments with a ligase), and amplifying the circularized fragments with rolling circle amplification, which comprises reverse transcription when in the context of an RNA nucleic acid.

Fragmenting a nucleic acid can be accomplished using chemical, mechanical or enzymatic methods. Preferably, fragmenting methods which reduce the amount of DNA damage is preferred. In some embodiments, the fragmenting comprises contacting the nucleic acid with a fragmentation enzyme. In select embodiments, the fragmentation enzyme is fragmentase, e.g., a nickase paired to or with an endonuclease. In some embodiment, the fragmentation enzyme is micrococcal nuclease (MNase). The methods may employ any concentration of fragmentation enzyme which results in efficient production of the desired size and quantity of fragments. For example, when using MNase, the methods may comprise 2 gel units per reaction (50 uL). The conditions for enzymatic fragmentation will depend on the enzyme utilized and the type and quantity of nucleic acid. In some embodiments, the reaction temperature of the fragmentation enzyme is less than 50 degrees Celsius. In some embodiments, the reaction temperature of the fragmentation enzyme is from 4 to 42 degrees Celsius. In some embodiments, the reaction temperature of the fragmentation enzyme is from 25 to 37 degrees Celsius. In some embodiments, the fragmenting does not include sonication.

Denaturing a nucleic acid can be accomplished using heat, alkali treatment, and salt. In some embodiments, the denaturing comprises treating the double stranded nucleic acid fragments with an alkaline agent, e.g., under conditions of alkaline pH. Alkaline pH generally capable of denaturing nucleic acids are pH values above about pH 9 (e.g., about 9.5, about 10, about 10.5, about 11, about 11.5, about 12, about 12.5, about 13, about 13.5, or about 14). In some embodiments, the alkaline pH is greater than 11. In some embodiments, the alkaline pH is about 12.5.

In some embodiments, the circularizing comprises contacting the single strand fragments with a ligase. In some embodiments, the ligase is an RNA ligase. In some embodiments, the circularizing is completed at a temperature of less than about 60 degrees Celsius (e.g., about 55° C., about 50° C., about 45° C., about 40° C., about 35° C., about 30° C., about 25° C., about 20° C., about 15° C., or about 10° C.). In some embodiments, the circularizing is completed at a temperature of about 10 to about 60° C., about 20 to about 50° C., about 20 to about 40° C., or about 20 to about 30° C. In some embodiments, the circularizing is completed at about 25° C. In some embodiments, the circularizing does not comprise contacting the single strand fragments with DNA repair enzymes.

In some embodiments, the methods further comprise treating double stranded nucleic acid fragments with a nucleotide kinase (e.g., to replace phosphates removed upon enzymatic fragmentation).

In some embodiments, the methods further comprise removing non-circularized single strand fragments prior to amplifying (e.g., enzymatically (e.g., exonuclease digest), by filtration or size separation).

In some embodiments, the methods further comprise extracting the nucleic acid from a source or sample. Preferably, the extraction is mild extraction facilitating preservation of nucleic acid sequence and structure. In some embodiments, the methods disclosed herein further comprise extraction of the nucleic acid without the use of high temperatures and/or phenol-chloroform extraction.

In general, fragmentation can be used to produce fragments of the nucleic acid of a specific size range. For example, the methods described herein can be used to generate fragments less than 250 basepairs for double stranded nucleic acids or 250 nucleotides for equivalent single stranded nucleic acid fragments. In some embodiments, the fragments are less than 150 basepairs or nucleotides. In some embodiments, the fragments are in the range of 25 to 150, 40 to 150, 50 to 150, 80 to 150, 100 to 150, 125 to 150, 25 to 100, 40 to 100, 50 to 100, 80 to 100, 25 to 80, 40 to 80, 50 to 80, 25 to 50, or 25 to 40 basepairs or nucleotides.

Rolling-circle amplification can be performed by a polymerase capable of strand displacement which repeatedly processes the circular template to synthesize a long, concatemeric strand. In some embodiments, the rolling circle amplification is primed with random primers. For example, random hexamers (NNNNNN) are hybridized with the circular fragments and the resulting double-stranded segments function as starting points for the polymerization reaction carried out by a polymerase, preferably with high strand-displacement activity such as phi29 DNA polymerase. As the extending complementary strand of the plasmid encounters double-stranded portions of nucleic acid, the advancing new strand displaces the old one from the template. This extension process covers the entire length of the circular DNA multiple times, resulting in the formation of repeated sequences, concatemers, of the template. The hexamers also hybridize with these concatemers, which then can become templates in their own right. The result is the formation of various lengths of double-stranded nucleic acids consisting of repeats of the template sequence.

In some embodiments, rolling-circle amplification can be performed by a polymerase capable of strand displacement, using a specific or series of specific primers rather than random hexamers. This could be useful for deep sequencing of plasmids, or targeted regions rather than whole genomes. Primer length is not limited to hexamers; primer length can be any size DNA, such as any primer between 6 and 60 nucleotides. There is no practical limit to primer length, although primer lengths beyond 30 nucleotides rapidly become redundant or superfluous.

In some embodiments, when the nucleic acid is RNA, the rolling circle amplification comprises rolling circle reverse transcription, following by amplification of the resulting repeated cDNA strands.

In some embodiments, the rolling circle amplification comprises incubating the circularized single strand fragments with a buffer having an EDTA concentration of 1 uM. In some embodiments, the rolling circle amplification lacks a primer annealing step. In some embodiments, the rolling circle amplification lacks DNA repair enzymes.

In some embodiments, the rolling circle amplification may occur at different temperatures. A range of temperatures from 4° C. to 37° C. have been used to successfully amplify ssDNA circles with rolling circle amplification.

The initial amount of isolated nucleic acid useful for the disclosed methods is less than 1000 ng. In some embodiments, the initial amount of isolated nucleic acid is 250 ng or less. In select embodiments, the initial amount of isolated nucleic acid is 125 ng or less. In some embodiments, the initial amount of isolated nucleic acid is at least 10 ng, at least 25 ng, at least 50 ng, or at least 75 ng.

The nucleic acid may be from any source. In some embodiments, the nucleic acid is isolated from a biological or living source. In some embodiments, the isolated nucleic acid is from a mammal, a plant, or a microorganism (e.g., virus, bacteria, parasite, fungus). In some embodiments, the nucleic acid is DNA. In some embodiments, the nucleic acid is RNA. In some embodiments, the nucleic acid is double stranded. In some embodiments, the nucleic acid is double stranded DNA.

In some embodiments, the DNA is genomic DNA from a subject. Genomic DNA may be from a biological sample, e.g., biological tissue, or body fluid from a subject that contains cells or free DNA. Genomic DNA may be from a microorganism. The genomic DNA may be from a single cell type or a heterogeneous sample of cells.

Methods for obtaining genomic DNA are also well known in the art. In certain embodiments, genomic DNA can be released and obtained by lysing cells from biological samples or single cells. Lysing may be performed using any suitable method known in the art, for example, lysing can be performed by means of thermal lysing, base lysing, enzymatic lysing, mechanical lysing, or any combination thereof.

Similar to extraction of the nucleic acid, it is preferred that lysis methods used with the disclosed methods are generally mild to avoid DNA damage. In certain embodiments, mild lysing methods may be used.

In some embodiments, the genomic DNA is obtained from a biological sample from a subject. In some embodiments, the subject is suspected or known to have a disease or disorder. In some embodiments, the genomic DNA is obtained from diseased or suspected diseased tissue or cell population. In some embodiments, the tissue or cell population is cancerous or suspected of being cancerous.

In some embodiments, the nucleic acid is obtained from a microbial organism. In some embodiments, the subject is suspected or known to have a disease or disorder. In some embodiments, the nucleic acid is obtained from diseased or suspected diseased tissue or cell population. In some embodiments, the tissue or cell population is cancerous or suspected of being cancerous.

In some embodiments, the methods further comprise sequencing the amplification products. Any sequencing method know in the art is useful with the disclosed methods e.g., next generation sequencing (NGS).

In some embodiments, the sequencing comprises preparing a library of amplification products, wherein each amplification product has at least one sequencing adaptor (e.g., at one or both ends of the amplification product). The adaptors may comprise a sequence of nucleotides for use in priming PCR or sequencing. Typical sequencing adaptors include (from 5′ to 3′) a first region, e.g., of about 10 to about 15, e.g., 12, nucleotides; a second region, e.g., of about 20 to about 60, e.g., 40, nucleotides that forms at least one (and preferably only one) hairpin loops and includes a sequence suitable for use in PCR priming and/or sequencing, e.g., next generation sequencing (NGS), flanked by at least one (and preferably only one) uracil; and a third region, e.g., of about 10 to about 15, e.g., 13, nucleotides that is complementary to the first region. The lengths of the first, second and third regions can vary depending on the sequencing method selected, as they are dependent on the sequences that are necessary for priming for use with the selected sequencing platform. In some embodiments, the sequencing further comprises amplifying and sequencing the library of amplification products.

In certain embodiments, products obtained from amplification using the method disclosed herein can be used to analyze genotypes or genetic polymorphisms in nucleic acids, such as single nucleotide polymorphism (SNP) analysis, short tandem repeat (STR) analysis, restriction fragment length polymorphism (RFLP) analysis, variable number of tandem repeats (VNTRs) analysis, complex tandem repeat (CTR) analysis, or microsatellite analysis and the like.

In certain embodiments, products obtained from amplification using the method disclosed herein can also be used for medical and/or diagnostic analysis. For example, a biological sample from an individual may be amplified using the method of the present application, and whether abnormalities such as mutations (e.g. substitutions, deletions, insertions) or fusion between chromosomes are present in gene or DNA sequence of interest in the amplification product can be analyzed, whereby to evaluate the risk of developing certain disease for the individual, the progression stage, genotyping and severity of the disease, or the likelihood that the individual respond to certain therapy. The gene or DNA sequence of interest can be analyzed using suitable methods known in the art, such as, but not limited to, nucleic acid probe hybridization, primer-specific amplification, sequencing a sequence of interest, single-stranded conformational polymorphism (SSCP), etc.

In some embodiments, the methods of the present application can be used to compare genomes derived from different cell populations. Such as between diseased or tumor cells and normal cells. Thus, in certain embodiments, the method of the present application can also further comprise analyzing the amplification product to identify disease- or phenotype-associated sequence features. In some embodiments, analyzing the amplification product comprises genotyping of DNA amplicon. In some other embodiments, analyzing the amplification product includes identifying polymorphism of DNA amplicons, such as single nucleotide polymorphism (SNP) analysis. SNP can be detected by some well-known methods such as oligonucleotide ligation assay (OLA), single base extension, allele-specific primer extension, mismatch hybridization and the like. A disease can be diagnosed by comparison of SNP to those of known disease phenotypes.

Also provided herein are kits for use in the methods described herein. The kits can include one or more of the following: a fragmentation enzyme, DNA polymerase, kinases (e.g., polynucleotide kinase), ligase (e.g., RNA ligase), nucleotides, exonuclease digest, reverse transcriptase, buffers, reagents useful for denaturation (e.g., alkali source), extraction, or lysis (e.g., detergents), and the like.

The kits can also comprise instructions for using the components of the kit. The instructions are relevant materials or methodologies pertaining to the kit. The materials may include any combination of the following: background information, list of components, brief or detailed protocols for using the compositions, trouble-shooting, references, technical support, and any other related documents. Instructions can be supplied with the kit or as a separate member component, either as a paper form or an electronic form which may be supplied on computer readable memory device or downloaded from an internet website, or as recorded presentation.

The kits provided herein are in suitable packaging. Suitable packaging includes, but is not limited to, vials, bottles, jars, flexible packaging, and the like. Individual member components of the kits may be physically packaged together or separately.

It is understood that the disclosed kits can be employed in connection with the disclosed methods. The kits may further contain containers or devices for use with the methods or compositions disclosed herein.

### 3. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Methods

Strain information. The E coli used herein is a descendent of the Foster lab's 2012 mutation accumulation experiment (Lee, H., et al, Proc. Natl. Acad. Sci. 109, E2774-E2783 (2012), incorporated herein by reference in its entirety). The Wild type “WT” genomic background is E. coli K-12 str. MG1655, and “MMR-” or mismatch-repair deficient, is derived from the WT strain by a mutL deletion, which causes E. coli to lose the ability to perform DNA replication mismatch surveillance. This results in a 100 to 150-fold increase in transition mutations (C:G→A:T, A:T→C:G). “S-” refers to strain 406-1, also known as MMR-L10-A1, an E. coli clone that emerged as a result of a long-term evolution experiment on the MMR-genetic background for 1000 days (See, Wei. W., et al., Nat. Commun. 13, 4752 (2022), incorporated herein by reference in its entirety). A mutation accumulation experiment was run on all strains, reaffirming/confirming their significantly different mutation rates. S- had the highest mutation rate measured at roughly 4×10⁻⁷, or roughly 2000-fold of the WT mutation rate.

Growth conditions and DNA extraction. E. coli were first grown overnight in liquid culture, (1×LB Broth, Miller) in borosilicate glass 10 mL tubes with loosely fitting metal caps from frozen stocks. The overnight culture was then serially diluted (100 uL culture to 900 uL PBS) in a biosafety cabinet to a range of dilutions around 1×10⁻⁷ and 1×10⁻⁸. Either 20 uL or 50 uL from each dilution was used to seed ten 10 mL tubes, with the aim of reaching a dilution rate of 0.5 bacteria per inoculum. When the correct dilution is hit, about half of the 10 tubes inoculated will become turbid overnight from E coli growth, and half will remain clear. Any ratio of ˜0.5 of 10 tubes or lower with E coli growth is indicative of growth from a single cell, under the assumption of a Poisson distribution of a discrete variable. E coli were grown at various intervals; 12 hours, from 10 pm to 10 am, 24 hours, from 10 am to 10 am, or 36 hours. Three ideal tubes (dilution 0.5 of tubes with growth or lower) from the serial dilution sets are chosen as biological replicates. CFUs are taken from the 10 mL tubes, and the rest of the culture is used for DNA extraction (Wizard Genomic DNA Extraction Kit, Promega), with the following protocol modifications: cells were heated only to 65° C. for 5 minutes with intermittent vortexing every minute to lyse cells, instead of 80° C. recommended by the protocol. Also, due to somewhat overloading the extraction protocol, 400 uL instead of 200 uL of the protein precipitation salt was used, and a 10 min spin of 16,000×G @room temperature was inserted post-precipitation to form a more solid pellet. The protocol otherwise followed the manufacturer specifications.

An additional test was performed on cells that grew as a colony on a plate after streaking, a single-cell bottleneck. These cells were then transferred into a LB tube for final bulk-up, in an attempt to measure the potential impact of environment on E coli mutation rates.

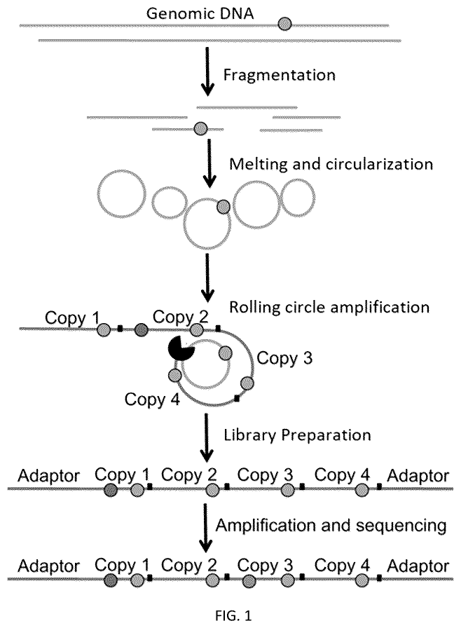

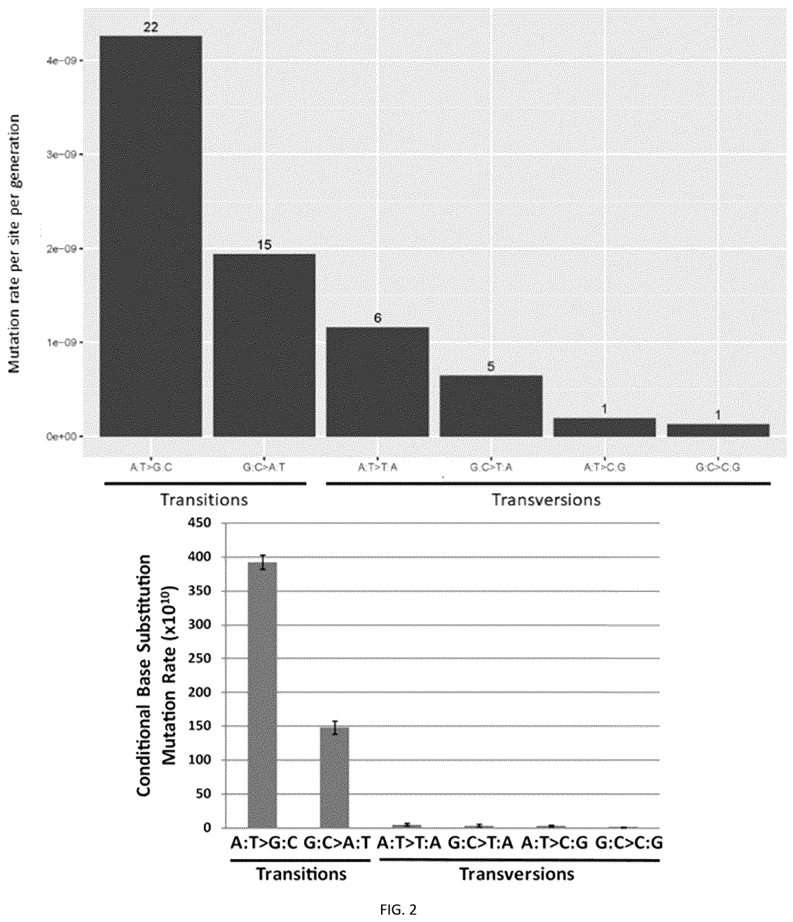

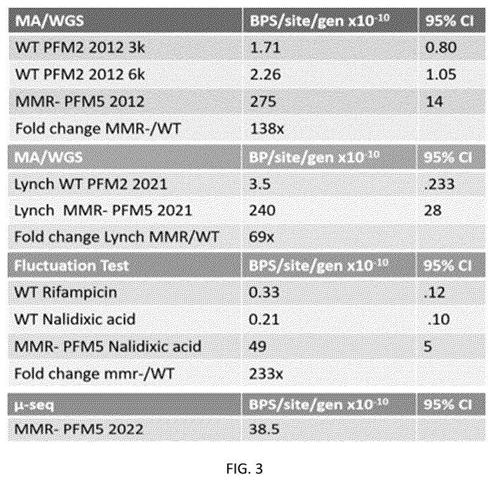

Library Preparation. Genomic DNA (gDNA) was then run through a gDNA cleanup column and eluted in autoclaved milli-q h20, pH 8, and stored at least overnight in a −80 freezer. DNA concentration was then measured via Qubit 4.0 with the 1× dsDNA HS kit; occasionally 1:10 dilutions were required to obtain a DNA concentration measurement, since the kit is most accurate at concentrations 10-40 ng/uL. Importantly, the 2-minute incubation before loading into the Qubit machine was found to be vital for measuring DNA concentration accurately and reproducibly over time. 125 ng of gDNA was then used as the input of the mu-seq protocol, see FIG. 1 for a full protocol. Briefly, the DNA is first fragmented with MNase (NEB), an enzyme with both endonuclease and exonuclease activity. Importantly, MNase is sensitive to degradation by vortexing; for a proper dilution, vortexing must be avoided. Instead, a 2 uL to 60 mL dilution was obtained by adding the Mnase to a bottle of water, pH 8, and inverting 20 times to mix before being put on ice. The enzyme does degrade in water over time, but it is not relevant over the time period of an afternoon. Sonication and/or other fragmentases may be used as MNase alternatives, but unless indicated otherwise MNase was ultimately used going forward.

After fragmentation and size verification the DNA is treated with phosphonucleotide kinase (PNK) due to the shearing mechanism of MNase leaving 3′ phosphates. The DNA is then melted with pH rather than heat; pH 12.5 is sufficient to turn double-stranded DNA into single-stranded DNA apparently without inducing DNA damage, whereas high temperatures are mutagenic. After DNA melting, the DNA is circularized to itself via RNA ligase I. No matter the concentration tested, self-ligation appears far more likely to occur than oligomer-ligation, although some degree of this is occurring. Circularization is followed by exonuclease digest, which destroys non-circularized DNA. After cleanup of the circularized DNA, rolling circle amplification is completed with phi29 and random hexamers.

Upon completion of the rolling-circle amplification portion of the mu-seq protocol, the existence of visible fine long strands of DNA in the PCR tube are an instant indicator of success or failure. After cleaning the DNA with a Zymo Oligo Clean and Concentrator, the DNA was assessed for quality control: fragment size, concentration, 260/230 ratio on the nanodrop. Routinely, 1-5 micrograms of DNA are produced from the protocol initiation of 125 ng, depending on the input DNA template and the fragment size. The DNA was either shipped to a sequencing core for library prep, or alternatively library construction was completed in-house. If in-house, the covaris ultrasonicator was used to shear the DNA to proper library sizes, followed by standard library prep following the NEBnext Ultra II protocol. Sequencing has been performed on the Illumina Hiseq 2500, Illumina Novaseq 6000, and BGI Americas sequencing platform DNB-seq. BGI Americas uses a pcr-free library prep kit for input amounts>1 microgram, which all libraries have met.

DNA extraction was modified to avoid high temperature and phenol-chloroform extraction. Additionally, all high temperature steps utilized to inactivate enzymes were removed. The input DNA amount was reduced from 2.5 micrograms to 250 ng, and then subsequently to 125 ng. The DNA fragmentation was changed from sonication to enzymatic fragmentation with MNase. The fragmentase reaction was improved which resulted in an increase in reliability/reproducibility of the protocol, by changing the dilution step of the manufacturer concentration, e.g., from 1 uL/1 mL to 2 uL/2 mL, significantly reducing pipettor error. The original protocol calls for a gel size selection after fragmentation; this was also avoided. The DNA was melted by pH instead of by temperature. The circularization step enzyme was changed from CircLigase II to RNA ligase I, although 3 microliters of ligase are added instead of 1 microliter; the circularization temperature dropped to 25° C. from 60° C. From the circularization reaction the DNA repair enzymes FPG and UDG were removed. The rolling circle amplification (RCA) temperature “primer annealing” step at 65° C. was also dropped. The RCA phi29 polymerase concentration was doubled, and the DNTP concentration increased 2.5×. Also, the quantity of EDTA in the 2× annealing buffer was reduced from 1 mM to 1 uM, significantly increasing yield. Post RCA, the reads were originally sequenced with the Hiseq 2500 with paired-end 250 bp reads, to good effect and additionally to the Novaseq 6000. However, to increase the flexibility of the assay to more platforms with higher yields, the assay was modified for PE150, using Beijing GenomicsInstitute's DNB-seq T7 platform.

The end result is a far leaner protocol that takes 1/20^{th} the input DNA, takes half the time to execute, produces micrograms of DNA for sequencing, and most importantly, significantly reduces the sequencing error rate encountered by the protocol.

Cell Division Count. Colony Forming Units (CFU's) were determined from 12 hour cultures by standard techniques, diluting to the 10⁻⁶ and 10-7 plates. Colonies were counted and an average was obtained. On average, approximately 28 cell divisions were responsible for the growth of the samples. However, because of filtering protocols, as described in the Data Analysis section, the first 5 cell divisions are discarded. Thus, the base-pair substitution per base sequenced (bps/base) rates were divided by 23 in order to obtain an estimate of bps/base/generation. These two measures are used interchangeably.

Data Analysis. A previously used pipeline was modified to measure RNA-seq reads generated by circle sequencing. Briefly, this pipeline started by identifying repeats within each read based on sequence similarity (minimum repeat size, 30 nucleotide (nt); minimum identity between repeats, 90%). Then, a consensus sequence of the repeat unit was built by summing the quality score of all four possible base calls (A, T, C, or G) from the repeats at each position and retaining the one with the highest total quality score. The next step consisted of identifying the position in the consensus sequence that corresponded to the 5′ end of the DNA fragment (because phi29 DNA polymerase is randomly primed, the concatemer of DNA, and therefore, the read sequence, can start anywhere on the circularized ssDNA). This was carried out by searching for the longest continuous mapping region in a BLAST mapping of a tandem copy of the consensus sequence against the reference genome. The consensus sequence was then reorganized to start from the identified ligation point (that is, the 5′ end of the original ssDNA fragment). This reorganized consensus sequence was then mapped against the genome with TopHat (version 2.1.0 with bowtie 2.1.0), and all nonperfect hits went through an algorithm of refining the search for the location of the ligation point before being mapped again. Finally, every mapped nucleotide was inspected and was retained only after passing a number of thresholds: (i) The mapped nucleotide must be supported by at least three repeats from the original sequence reads, (ii) all repeats must support the same base call, (iii) the sum of base call qualities at this position is above 100, (iv) the nucleotide must be more than 5 nt away from the end of the consensus sequence (to minimize false-positives induced by mapping errors), and (iv) the nucleotide must also be at a genomic position covered by at least 20 reads and with less than 5% of these reads supporting a base call different than that of the reference genome (this allows for filtering out polymorphic sites). For each read containing at least one mismatch passing these thresholds, sequences corresponding to all possible versions of the position of the ligation point were generated and mapped against the genome with TopHat. If at least one of these sequences finds a perfect match, then the original read is discarded. This last test removes a small fraction of the error-containing reads (typically less than 5%), but it ensures that errors in calling the position of the ligation point cannot produce false-positives. Every mapped nucleotide that passes all these thresholds was considered as an event of transcription for which the transcribed nucleotide was known with certainty, and the total transcription error rate was calculated as the number of mismatches divided by the total number of mapped nucleotides that passed all quality thresholds. Put another way, every mapped nucleotide that passes all these thresholds was considered as an event of DNA mutation, and the total DNA mutation rate was calculated as the number of unique mismatches divided by the total number of mapped nucleotides that passed all quality thresholds. Because some mutations will arise “early” in the growth of the sample, and are known as jackpot mutations, the infinite sites assumption that any mutation identified more than once arose from a single mutation event was assumed.

### Example 1

Mu-seq is capable of reproducing the mutation rate of MMR-E coli as measured by fluctuation tests, rather than the mutation rate of MMR-E coli as measured by mutation accumulation/whole genome sequencing (MA/WGS). Using the methods for mu-seq disclosed herein, a mutation rate of 3.85×10⁻⁹ base pair substitutions per site per generation (bps/site/gen) was identified, from a raw error rate of 1.16×10⁻⁷ per base sequenced. 3.85×10⁻⁹ is similar to the estimate of MMR-fluctuation tests, of ˜4.99×10⁻⁹ bps/site/gen, although less than the mutation rates measured in MA experiments for the same bacterial clone by about 7.3-fold. The general fold-variation reported from fluctuation tests and MA/WGS experiments is between 6-9-fold in WT E. coli.

The mutation spectrum of mu-seq is also quite similar to that of the MA experiment, save that there are fewer mutations detected. In general, there is an abundance of transition mutations relative to transversion mutations, and furthermore the ratio of AT→GC and GC→AT is quite similar; the published ratio is 2.6:1, and the ratio called by mu-seq is 2.31:1. The resemblance of the spectrum and the mutation rate of the fluctuation test provide two independent means of increasing confidence in the accuracy of the experiment. This indicates that mu-seq is capable of accurately measuring the mutation rate of MMR-, unmatched by any single-sample sequencing technology to date. There is a relative increase of AT>TA and GC>TA transversions above proportions expected from MA experiments, however, there are no mutation spectrums from fluctuation tests against which this may be compared. As shown with the above data, the disclosed methods result in a sequencing protocol which drops the resolution floor between 76× and 1750×, depending on the baseline number used for comparison from the previous circularizing sequencing protocol.

### Example 2

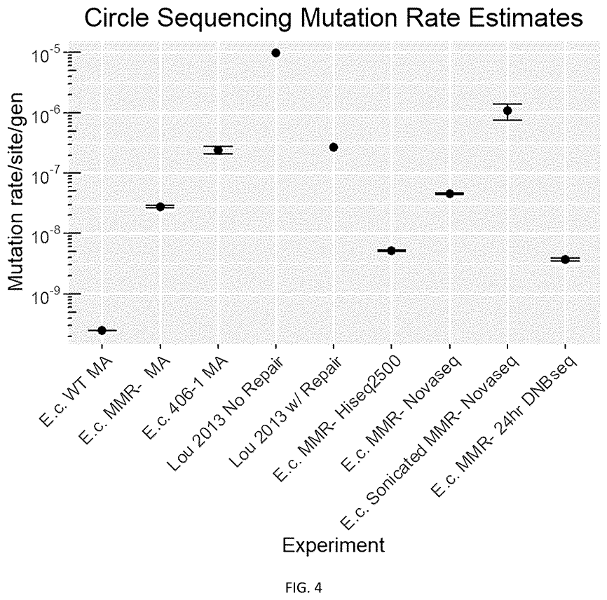

The mutation rates of wild-type, MMR-, and 406-1 E. coli were used as a sort of molecular ruler or ladder, on which DNA RCA results may be measured. FIG. 4 demonstrates that although the 2013 results are improvements over traditional sequencing error rates, the previous rates do not come close to describing the WT yeast DNA mutation rates on whose DNA the technique was developed. The yeast mutation spectrum is also relatively flat, and the mutation spectrum results from 2013 did not provide much insight as to whether the protocol was truly working. A pilot spike-in experiment of WT E. coli reproduced an identical mutation rate to Lou et al 2013, indicating modifications to the protocol would ensure more accurate mutation rate determinations.

To reduce the amount of DNA damage incurred by the protocol, various methodological modifications, as described above in Methods: Library Preparation, were introduced. Mismatch repair deficient (MMR-) E. coli was chosen as a primary model to measure mutation rates, as somewhat of a “positive control” with a relatively high mutation rate that grows overnight. A first attempt on the Hiseq-2500 machine yielded promising results, though the mutation rate estimate per generation appeared too low. Changing to Novaseq 6000 platform for increased sequencing yield, and confirmed initial data suggesting that sonication is indeed mutagenic. The exact same genomic DNA from which circle-seq libraries were made by enzymatic fragmentation were then used for sonication; these resulted in elevated mutation rate estimates (FIG. 4).

However, the Novaseq run had MMR-mutation rates estimates that were higher than the initial Hiseq-2500 run. This may be due to problems with Novaseq on the PE250 setting, or it might be due to poor library preps. Another sequencing platform was used to identify which rate ought to be more consistent, and furthermore, transfer from PE250 sequencing reads to PE150 sequencing reads. Loading the Circle-sequencing libraries onto a DNB-seq machine produced a new set of estimates, which most closely matched the original Hiseq-2500 reads; with the limited sample size they are not statistically different from one another (Table 1). Overall, the mutation rate estimates of circle-sequencing remain 6 to 8-fold lower than the mutation rates estimated by mutation accumulation experiments.

TABLE 1
Mutation Rate Estimation Summary

| | bps/ | bps/base/ | Std. | | |
|---|---|---|---|---|---|
| | base × | gen × | dev. × | S.E.M. × | p. val vs. |
| Library | 10⁻⁷ | 10⁻⁹ | 10⁻⁸ | 10⁻¹⁰ | DNB-seq |
| Hiseq2500 | 1.22 | 5.30 | 9.82 | 24.7 | 1* |
| MMR- | | | | | |
| Novaseq | 5.30 | 23.0 | 4.36 | 1.09 | <2.2 × 10⁻¹⁶* |
| MMR- | | | | | |
| Novaseq | 230 | 1000 | 1000 | 3,971 | 0.002** |
| MMR- +S | | | | | |
| Novaseq | 8.59 | 37.3 | 21.5 | 66 | 0.63*** |
| 406-1 | | | | | |
| Novaseq | 253 | 1100 | 360 | 767 | <2.2 × 10⁻¹⁶* |
| 406-1 +S | | | | | |
| DNBseq | 1.03 | 4.5 | 1.17 | 2.94 | 1 |
| MMR- | | | | | |

base-pair substitutions. gen: generations. S.E.M: Standard Error of the Mean. +S indicates the sample was sonicated for initial genomic DNA fragmentation.
P values:
*Bonferroni corrected z-test;
**Tukey test;
***Bonferroni-corrected t-test.

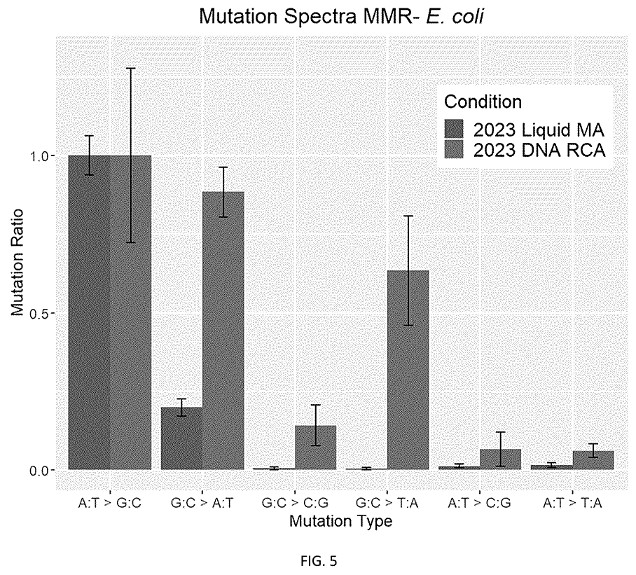

To better understand the mutation rate estimates, the spectrum of DNA mutations generated in the growth experiment was investigated. To better compare like-for-like experiments, a mutation accumulation experiment in liquid culture was run (See, S. Baehr, et al., bioRxiv 2023.08.31.555790; doi.org/10.1101/2023.08.31.555790). This assay ensured that environmental growth conditions should not be a cause for any perceived differences in mutation spectrum. Direct comparison of the Liquid MA spectrum to DNA rolling circle data (FIG. 5) utilized normalization of the ratios of the rates, as the mutation rates themselves are 6 to 8-fold different. In the ratio comparison the spectra are clearly not identical. Specifically, although there is a signal of elevated A:T→G:C, the DNA RCA protocol has additional spectral contributions to an overall mutation rate, all related to G:C base pair instability. These mutation spectra are similar to those seen from artifacts of oxidative DNA damage, from 8-oxo-guanosine and from cytosine deamination. Therefore, despite reporting a potentially lower mutation rate, it appears the mutation spectrum is detecting some degree of in vitro noise, on the order of two-thirds of base substitutions detected not conforming to the expected mutation spectrum. Literature estimations of emph in vitro DNA damage are on the order of 1×10⁻⁶ per base (L. D. Scanlan, et al., Analytical Chemistry, 91(19):12149-12155, October 2019). The published mutation rate of MMR-E. coli is 3×10⁻⁹, about a third of the rate detected, though the mutation rate per base may be slightly different from the standard units obtained from a mutation accumulation experiment, mutation rate/site/gen.

Despite the apparent limitations of this iteration of circle-sequencing, many in vitro sources of DNA damage can be quantified. Compared to the original circle-sequencing protocol absent DNA repair enzymes, the resolution of sequencing in the protocol used herein has increased by over 1000-fold. Many sources of in vitro DNA damage are known and have been quantified, from the high heat of the melting cycle of PCR to the contribution of phenol in phenol-chloroform methods of DNA extraction.

Covaris ultrasonication by the S220 machine is immensely appealing as a means of fragmentation in the assembly of library preps. Unlike enzymatic fragmentation, which is very sensitive to starting DNA amount, ultrasonication shears DNA reliably somewhat invariant of starting concentration. Library preps of the exact same genomic DNA samples were prepared using enzymatic fragmentation and ultrasonication. This direct comparison was made for both a MMR-strain of E. coli, and additionally a hypermutator strain of E. coli, 406-1. For the purpose of shearing dsDNA to 50-125 base pairs (as for enzymatic fragmentation), ultrasonication of 50 minutes was used, a timeframe that exceeds most conventional ultrasonication protocols.

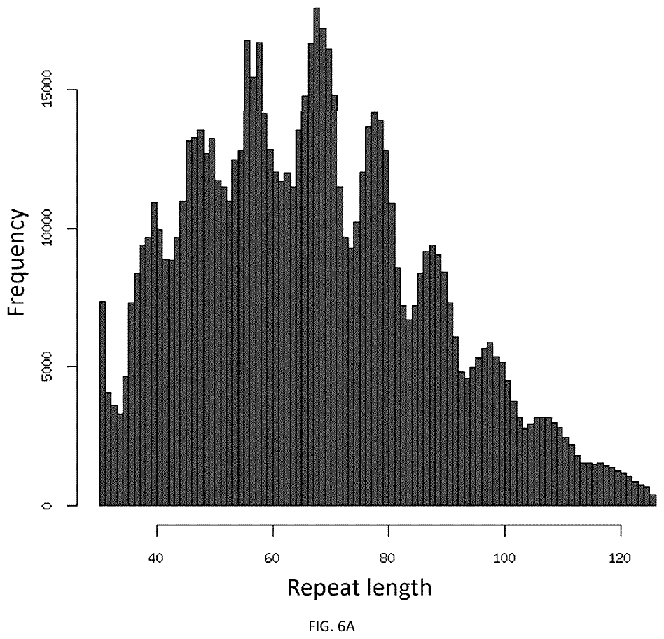

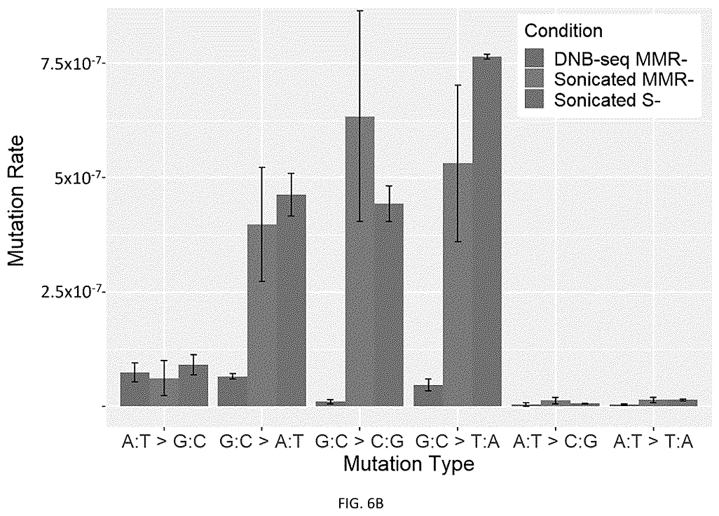

The initial alignment of repeats produces a striking sawtooth pattern of repeat sizes (FIG. 6A). This sawtooth pattern with a periodicity of 10 bp must allude to ssDNA's circularization efficiencies, rather than sonication itself. Enzymatic fragmentation demonstrates considerably more bias in fragment size generation. As shown in Table 1, the samples with sonication (+S) have an increased mutation rate estimate, on the order of 100-fold.

The mutation spectrum change is shown in FIG. 5B. A quantification of the mutation spectrum of sonicated samples demonstrates the impact of sonication upon DNA. The same mutations that were increased in abundance in circle-seq relative to the liquid MA are apparent, but are now the entirely dominant forms of mutation. The G:C base appears susceptible to sonication damage. The evidence of this experiment suggests that the DNA damage burden of sonication is at most on the order of 1 in 100,000 bases.

What is claimed is:

1. A method for amplifying nucleic acids, comprising:
fragmenting isolated nucleic acids to generate a plurality of nucleic acid fragments;
circularizing the plurality of fragments with a ligase; and
amplifying circularized single strand fragments with rolling circle amplification,
wherein when the nucleic acid is double stranded the method further comprises denaturing the isolated nucleic acids or the plurality of nucleic acid fragments prior to the circularizing, and
wherein the method produces at least about 1 microgram of amplification products at a mutation rate with a sequencing resolution floor of at least about 1×10⁸ per base.

2. The method of claim 1, wherein the fragmenting comprises contacting the isolated nucleic acids with a fragmentation enzyme.

3. The method of claim 2, wherein the fragmentation enzyme is micrococcal nuclease (MNase).

4. The method of claim 1, wherein the denaturing comprises treating the plurality of double stranded nucleic acid fragments under conditions of alkaline pH.

5. The method of claim 4, wherein the alkaline pH is about 12.5.

6. The method of claim 1, wherein the circularizing is completed at 25° C.

7. The method of claim 1, wherein the circularizing does not comprise contacting the plurality of single strand fragments with DNA repair enzymes.

8. The method of claim 1, wherein the method does not comprise size selection of the plurality of fragments.

9. The method of claim 1, further comprising at least one or all of:
treating the plurality of nucleic acid fragments with a nucleotide kinase;
removing non-circularized fragments prior to amplifying; and
extracting the nucleic acid without high temperature or phenol-chloroform extraction.

10. The method of claim 1, wherein the nucleic acid fragments are less than 150 basepairs (bp) or nucleotides (nt).

11. The method of claim 1, wherein the rolling circle amplification is primed with random primers.

12. The method of claim 1, wherein the rolling circle amplification comprises a buffer having an EDTA concentration of 1 uM.

13. The method of claim 1, wherein the rolling circle amplification lacks a primer annealing step and/or DNA repair enzymes.

14. The method of claim 1, wherein the isolated nucleic acid is present at an amount of about 125 ng.

15. The method of claim 1, wherein the isolated nucleic acid is DNA.

16. The method of claim 15, wherein the DNA is obtained from a subject or microorganism.

17. The method of claim 15, wherein the DNA is obtained from a diseased or suspected of being diseased tissue or cell population.

18. The method of claim 1, further comprising sequencing the amplification products.

19. The method of claim 18, wherein the sequencing comprises preparing a library of amplification products, wherein each amplification product has at least one adaptor, and amplifying and sequencing the library of amplification products.

20. The method of claim 18, further comprising: detecting nucleotide variations or mutations in the amplification products or the library of amplification products; identifying disease- or phenotype-associated sequence variations; determining mutation rates; or a combination thereof.
